(19) United States
(12) Patent Application Publication
(10) Pub. No.: US 2023/0296384 A1
(43) Pub. Date: Sep. 21, 2023

(54) INFORMATION PROCESSING DEVICE, INFORMATION PROCESSING METHOD, AND PROGRAM

(71) Applicant: CASIO COMPUTER CO., LTD., Tokyo (JP)

(72) Inventor: Futoshi YAMAMOTO, Tokyo (JP)

(73) Assignee: CASIO COMPUTER CO., LTD., Tokyo (JP)

(21) Appl. No.: 18/109,348

(22) Filed: Feb. 14, 2023

(30) Foreign Application Priority Data
Mar. 18, 2022 (JP) 2022-044271

(51) Int. Cl. G01C 21/20

(52) U.S. Cl. CPC G01C 21/20; G16H 20/30

(57) ABSTRACT

An information processing device includes a route information acquisition unit configured to acquire route information that is information on a route along which a user performs at least two types of moving with different moving speeds, and a section setting unit configured to set, on the route based on the acquired route information, a first section as a section in which the user performs first moving at a first speed and a second section as a section in which the user performs second moving at a second speed lower than the first speed.

### CROSS-REFERENCE TO RELATED APPLICATIONS

This application is based on and claims the benefit of priority from Japanese Patent Application No. 2022-044271, filed on Mar. 18, 2022, the content of which is incorporated herein by reference.

### BACKGROUND

### Technical Field

The present disclosure relates to an information processing device, an information processing method, and a program.

### Related Art

Walking is an example of exercise that can be performed without difficulty to maintain and improve health. In particular, attention has been paid to an exercise method called interval walking in which walking at a normal pace and walking at a faster pace than normal are alternately performed to enhance exercise effects. Interval walking can be performed in various scenes and various routes, for example, parks and streets.

On the other hand, there has been proposed a technique for automatically generating a route when a user performs training such as running. For example, Japanese Unexamined Patent Application, Publication No. 2020-58821 discloses a technique in which a popularity level of activity and an activity level are determined based on various activity data of a user and displayed on a map, and a route passing through a region indicating the activity on the map selected by the user is automatically generated.

However, in the case of interval walking described above, since a repetition cycle of walking at a normal pace and walking at a fast pace is set based only on a predetermined time, sections such as sharp curves or steep slopes, which are not suitable for fast walking on a route, may be set as fast walking sections, and a walking program bringing about appropriate exercise effects for the user may not necessarily be provided. Japanese Unexamined Patent Application, Publication No. 2020-58821 also does not disclose a process of reflecting curves and gradients included in the route itself in automatic setting of the route.

### SUMMARY

An aspect of the present disclosure provides an information processing device including:
a route information acquisition unit configured to acquire route information that is information on a route along which a user performs at least two types of moving with different moving speeds; and
a section setting unit configured to set, based on the acquired route information, the route to a first section as a section in which the user performs first moving at a first speed and a second section as a section in which the user performs second moving at a second speed lower than the first speed.

Further features of the present invention will become apparent from the following description of exemplary embodiments (with reference to the attached drawings).

### BRIEF DESCRIPTION OF THE DRAWINGS

FIG. 1 is a schematic diagram illustrating a standard walking pattern according to an embodiment;

FIG. 2 is a system configuration diagram illustrating a system configuration of an exercise assistance system according to an embodiment;

FIG. 3 is a block diagram illustrating a hardware configuration of an exercise assistance device according to an embodiment;

FIG. 4 is a block diagram illustrating functions implemented by a processing unit of the exercise assistance device according to an embodiment;

FIG. 5 is a schematic diagram showing an example of a moving route according to an embodiment in a horizontal line shape;

FIG. 6 is a schematic diagram showing an example of a moving route according to an embodiment in a vertical line shape;

FIG. 7 is a schematic diagram showing an output display example of a walking pattern created by the exercise assistance device according to an embodiment;

FIG. 8 is a flowchart illustrating a walking pattern setting process according to an embodiment;

FIG. 9 is a flowchart illustrating a walking pattern modification process according to an embodiment;

FIG. 10 is a schematic diagram showing an example of a moving route according to another embodiment in a horizontal line shape;

FIG. 11 is a schematic diagram showing an example of a moving route according to another embodiment in a vertical line shape;

FIG. 12 is a schematic diagram showing an output display example of a moving pattern. created by the exercise assistance device according to another embodiment; and

FIG. 13 is a flowchart illustrating a moving pattern setting process according to another embodiment.

### DESCRIPTION OF THE EMBODIMENTS

Hereinafter, embodiments of the present disclosure will be described with reference to the accompanying drawings.

### First Embodiment

### <Interval Walking>

As described above, interval walking is an exercise method. in which a walk at a normal speed and a fast walk at a faster speed than the normal speed are repeated for a predetermined period of time. An academic evidence for this is disclosed in, for example, “Hiroshi Nose et al., “Effects of Interval Walking Training on Lifestyle-related Diseases and Physical Fitness in Middle-Aged and Older People—Current status and future of Matsumoto City Junior College of Physical Education—”, Physical Therapy, Vol. 36, Issue 4, pages 148-152, 2009.”

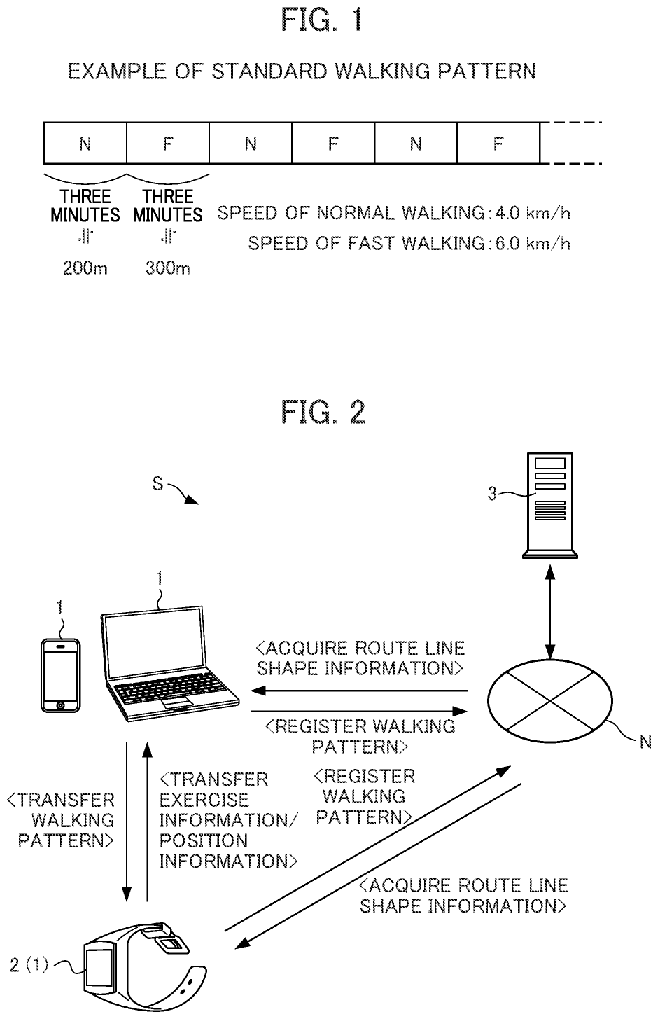

According to the same paper, as interval walking®, an exercise method is introduced in which, for a peak oxygen uptake (VO2ₚₑₐₖ) determined by a prescribed test, fast walking at a speed exceeding 70% of the peak oxygen uptake and normal walking at a speed not exceeding 30% of the peak oxygen uptake are alternately repeated every three minutes. Since there are individual differences in peak oxygen uptake, it is not possible to uniformly define the speed of fast walking and normal walking, but, for the sake of convenience, the description will be made herein assuming that the speed of normal walking is 4.0 km/h and the speed of fast walking is 6.0 km/h. When such a walking pattern is referred as a “standard walking pattern” in which the normal walking and the fast walking are alternately repeated every three minutes, a pattern is repeated in which a walking distance during the normal walking is 200 m and a walking distance during the fast walking is 300 m. FIG. 1 schematically shows an example of the standard walking pattern. In FIG. 1, a symbol N indicates normal walking, and a symbol F indicates fast walking.

### <Outline of System Including Exercise Assistance Device>

FIG. 2 is a system configuration diagram illustrating a system configuration of an exercise assistance system S including an exercise assistance device according to an embodiment of the present disclosure. As shown in FIG. 2, the exercise assistance system S includes an exercise assistance device 1 implemented by an information processing device such as a smart phone or a personal computer, and a terminal device 2 such as a smart watch. The exercise assistance device 1 is communicably connected to a server computer 3 via a communication network such as the Internet, LAN, or WAN. The exercise assistance device 1 and the terminal device 2 are communicably connected to each other by wireless communication such as Bluetooth®.

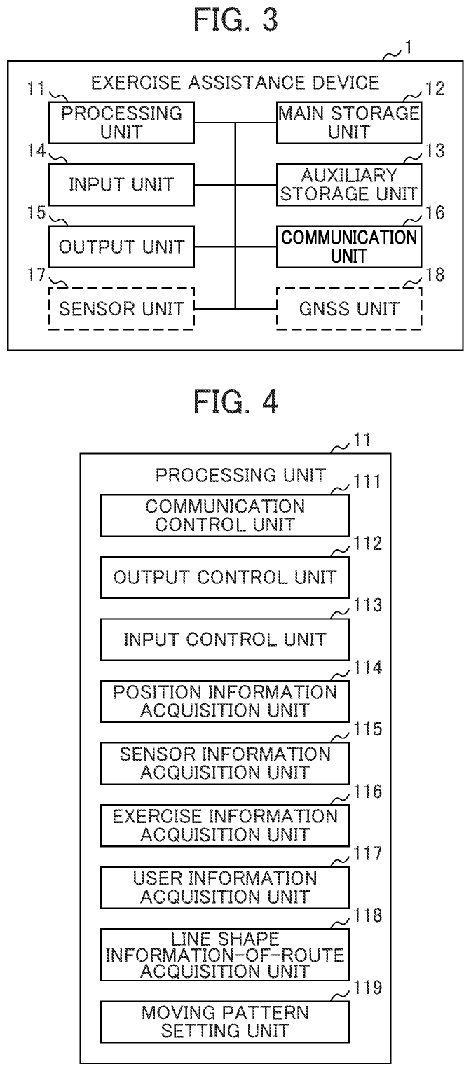

The exercise assistance device 1 acquires route line shape information (route information) regarding a route, on which a user intends to perform interval walking, from an external device through a communication network N, for example, and generates, based on user information input by the user, a walking pattern suitable for the user to perform interval walking on the route in consideration of the route line shape information. The “route line shape information” is information on gradients and curves in the route that user intends to walk. The route line shape information may include information on a road condition or other information on a position of a traffic light in a case of urban routes. The exercise assistance device 1 can register the generated walking pattern in the server computer 3. The registered walking pattern may be shared by a plurality of users. A route can be set in such a manner that a user accesses map data through the smart phone as the exercise assistance device 1 and inputs a route on the map. Alternatively, a route may be set in such a manner that the user selects from a plurality of model routes that are registered in the server computer 3 or another external device in advance. Data of the line shape of the route can be acquired from the map data, as a horizontal line shape and altitude information as a set of position coordinates along the set route. When the exercise assistance device 1 is implemented as a smart phone, the user can attach the smart phone to their body and use it as a terminal device 2 to be described below. The exercise assistance device 1 can be implemented as various forms of devices. As a form of the exercise assistance device 1 according to the present embodiment, a configuration example shown in FIG. 3 will be described below.

The terminal device 2 is generally a mobile terminal device such as a smart watch worn by a user who intends to execute a walking exercise, and is communicably connected to the exercise assistance device 1 via a communication means such as Bluetooth. In the present embodiment, for example, the terminal device 2 has a function of transferring and outputting walking pattern data on the walking route created by the exercise assistance device 1 or a function of transferring information indicating a state of the user, who is detected by various sensors provided in the terminal device 2, to the exercise assistance device 1. The terminal device 2 is configured to be able to communicate with the server computer 3 via the communication network N, and thus can also be configured to function as the exercise assistance device 1. In this case, the terminal device 2 functioning as the exercise assistance device 1 acquires the route line shape information from the communication network N to create a walking pattern, and functions to register the created walking pattern in the server computer 3 via the communication network N.

The server computer 3 is a computer that manages functions of the exercise assistance system 3, and has a function of registering and managing walking pattern data for each user for each of routes created by the respective exercise assistance devices 1 by providing the information on the user necessary for creating the walking pattern and the route line shape information to the exercise assistance device 1. The server computer 3 may be a computer installed at one location, or may be configured as a cloud system configured by a plurality of computers distributed over a network.

### <Configuration Example of Exercise Assistance Device>

A configuration of the exercise assistance device 1 according to the present embodiment will be described below. FIG. 3 is a block diagram illustrating a hardware configuration of the exercise assistance device 1 according to the embodiment of the present disclosure. As shown in FIG. 3, the exercise assistance device 1 of the present embodiment includes a processing unit 11, a main storage unit 12, an auxiliary storage unit 13, an input unit 14, an output unit 15, and a communication unit 16. A sensor unit 17 and a GNSS unit 18 indicated by dashed lines in FIG. 3 are not essential as hardware components of the exercise assistance device 1.

As described with reference to FIG. 2, the exercise assistance device 1 of the present embodiment can be implemented in various forms. A main function of the exercise assistance device 1 is to acquire route information including curve and gradient information on a route along which the user intends to perform moving, to generate a moving pattern in consideration of the route information, and to provide it to the user. Therefore, the exercise assistance device 1 of the present embodiment can first be configured as an information processing device such as a smart phone, a tablet terminal, or a personal computer that does not include the sensor unit 17 and the GNSS unit 18. In this case, the server computer 3 illustrated in FIG. 2 can also be configured to function as the exercise assistance device 1. The exercise assistance device 1 configured in this manner can generate a moving pattern suitable for the route, on which the user performs moving, based on the route information acquired from the outside, and provide the moving pattern to the user. The exercise assistance device 1 of the present embodiment can also be configured as an information processing device including the sensor unit 17 and the GNSS unit 18. In this case, the exercise assistance device 1 can be implemented, for example, as a smart phone or a wristwatch type terminal including all the hardware components shown in FIG. 3, and enables creation of a walking pattern based on the route line shape information in a scene in which the user performs exercise. In particular, a wearable terminal such as a smart watch as the exercise assistance device 1 can be used to create a walking pattern based on the route line shape information in a worn state during exercising, which improves convenience for the user. As will be described below, the exercise assistance system S can be used not only for the user to create the walking pattern, but also to create the moving pattern during the moving in which walking and running are combined.

Each of the hardware components illustrated in FIG. 3 will be described. The processing unit 11 is configured by a processor that performs various arithmetic operations and control processes required for the operation of the exercise assistance device 1. The processing unit 11 may also be called a processor. Examples of the processor constituting the processing unit 11 include a CPU (Central Processing Unit), an MPU (Micro Processing Unit), an SoC (System on a Chip), a DSP (Digital Signal Processor), a GPU (Graphics Processing Unit), an ASIC (Application Specific Integrated Circuit), a PLD (Programmable Logic Device), or an FPGA (Field-Programmable Gate Array) and combinations thereof. Further, the processing unit 11 may be a combination of these processor with a hardware accelerator or the like.

The main storage unit 12 stores programs such as firmware, system software, and application software, and also functions as a work area that is temporarily used when various processes are performed. The main storage unit 12 includes, for example, a ROM (Read Only Memory) serving as a non-volatile memory and a PAM (Random Access Memory) serving as a volatile memory.

The auxiliary storage unit 13 stores user information, the acquired route line shape information, the created walking pattern information, and the like. The auxiliary storage unit 13 includes a semiconductor memory or the like.

The input unit 14 includes, for example, a touch panel, various buttons such as keys, and a microphone, and receives an operation from a user. The output unit 15 includes a display on which the above-described touch panel is mounted to display an image and a speaker for amplifying sounds, and outputs images and sounds.

The communication unit 16 controls communication between the exercise assistance device 1 and the external information processing device such as another terminal device 2 or the server computer 3. The communication unit 16 includes, for example, network connection devices of SIM (Subscriber Identity Module) cards and network adapters, and wireless communication devices based on communication standards of ELF (Bluetooth® Low Energy), Wi-Fi® (Wireless Fidelity), or NEC (Near Field Communication).

The communication unit 16 may adopt a method of exchanging information with an external information processing device via a network such as the Internet, or may adopt a method of exchanging information with an external information processing device through an information processing terminal to be paired.

The sensor unit 17 includes various sensors that detect state and movement of the user. The sensor unit 17 includes, for example, an acceleration sensor, an angular velocity sensor, a geomagnetic sensor, a pressure sensor, and a heartbeat sensor. The acceleration sensor, the angular velocity sensor, the geomagnetic sensor, and the pressure sensor may cause the sensor unit 17 to function as a behavior detection unit for detecting the movement of the user or to function as a position specifying unit together with the GNSS unit 18 to be described below. In addition, the heartbeat sensor can cause the sensor unit 17 to function as a biometric information acquisition unit for acquiring a heart rate of the user as biometric information.

The GNSS unit 18 is a positioning information acquisition unit for acquiring position information. GNSS is an abbreviation for Global Navigation Satellite System, and the GNSS unit 16 is a satellite positioning device that uses a satellite positioning system such as GPS (Global Positioning System). The GNSS unit 18 is configured by an antenna and electronic components to acquire positioning satellite signals transmitted from a plurality of positioning satellites and to specify its own position.

Next, functions implemented by the processing unit 11 of the exercise assistance device 1 will be described. FIG. 4 is a block diagram illustrating functions implemented by the processing unit 11 of the exercise assistance device 1 according to an embodiment of the present disclosure. The processing unit 11 of the present embodiment includes a communication control unit 111, an output control unit 112, an input control unit 113, a positon information acquisition unit 114, a sensor information acquisition unit 115, an exercise information acquisition unit 116, a user information acquisition unit 117, a route line shape information acquisition unit 118, and a moving pattern setting unit 119.

The communication control unit 111 executes a process for the exercise assistance device 1 to communicate with external devices via the communication unit 18. For example, the communication control unit 111 communicates with the terminal device 2 or the server computer 3 to transmit and receive various information.

The output control unit 112 executes a process of displaying an image on a screen of the output unit 15. For example, the output control unit 112 executes a process of displaying a walking pattern display image of a certain walking route created by the exercise assistance device 1 on the screen of the output unit 15.

The input control unit 113 executes a process of accepting an operation of the input unit 14 by the user. For example, the input control unit 113 executes a process of accepting an input operation executed by the user via the input unit 14 based on an operation screen displayed on the screen of the output unit 15.

The position information acquisition unit 114 has a function of calculating, as position information, a latitude, a longitude, and an altitude indicating a present position of the user who wears the exercise assistance device 1 or the terminal device 2 including the sensor unit 17 and the GNSS unit 18, based on the positioning satellite signal received by the GNSS unit 18 and the pressure signal obtained from the pressure sensor of the sensor unit 17.

The sensor information acquisition unit 115 has a function of acquiring measurement values obtained by each sensor from sensor signals acquired by various sensors provided the sensor unit 17. The sensor unit 17 may be provided in the exercise assistance device 1, or may be provided in an external device such as a wearable terminal that cooperates with the exercise assistance device 1. This is the same for the exercise information acquisition unit 116 to be described below.

The exercise information acquisition unit 116 has a function of detecting movement of the exercise assistance device 1, based on the measurement values obtained through the sensor information acquisition unit 115 from the acceleration sensor, the angular velocity sensor, the geomagnetic sensor, and the like of the sensor unit 17.

The user information acquisition unit 117 has a function of acquiring user information input by the user via the input unit 14 and storing the user information in the auxiliary storage unit 13. The user information can include, for example, an ID that is user identification information, and user attribute information such as age, gender, height, and weight. Based on the user information, the exercise assistance device 1 calculates the peak oxygen uptake or the maximal oxygen uptake of the user and records it as user information. The user information is used as data for the exercise assistance device 1 to create a walking pattern with a high exercise effect for each user and for each route. The user information may be registered in the server computer 3 in advance to be read by the smart phone, the personal computer, the smart watch, or the like serving as the exercise assistance device 1 as necessary when the user creates a walking pattern.

The route line shape information. acquisition unit 118 has a function of acquiring line shape information on a walking route for which a walking pattern is to be created from the external device such as the server computer 3. Here, the “line shape information” refers to information in which a starting/ending point and a curve radius of a curved section and a starting/ending point and an inclination angle of a gradient section are recorded with respect to the position on the route from a starting point to an ending point of the route along which walking exercise is performed. When creating a walking pattern for each route, the exercise assistance device 1 of the present embodiment creates walking pattern using whether curves and gradients on the route are suitable for fast walking as a determination reference, in addition to the allocation between fast walking and normal walking. In addition to curves and gradients, the route line shape information may include places of interest (high spots) (Point Of Interest, POI) on the route. In addition, the route line shape information may appropriately include other information that serves as a reference for determining the walking pattern when interval walking exercise is performed on the route.

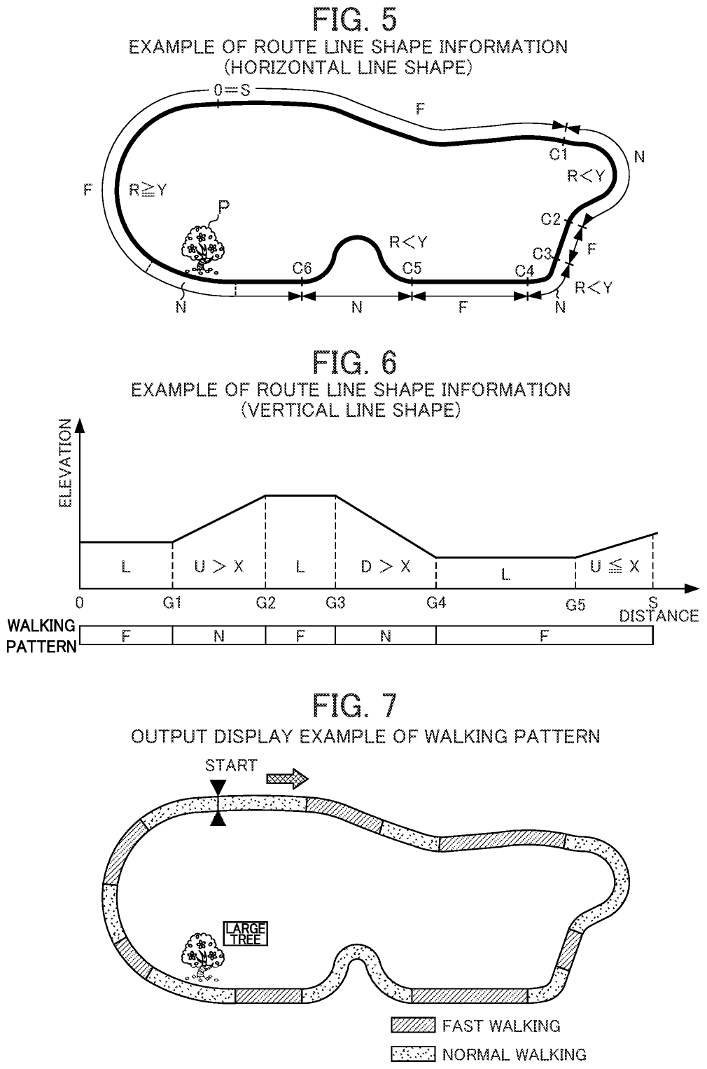

FIG. 5 is a schematic diagram of a horizontal line shape showing a relation between a position on a route and a curve for a certain walking route, and FIG. 6 is a schematic diagram of a vertical line shape showing a relation between a position on a route and a curve for the same walking route. Referring to FIG. 5, the exemplary route is a circular route, and is generally configured by a combination of a straight section, gently curved section, and a sharply curved section. The circular route illustrated in FIG. 5 has the following horizontal line shape starting clockwise from a spot (a distance being s=0) as a starting point.

| Section on route | Curve radius R |
|---|---|
| 0-C1 | R ≥ Y |
| C1-C2 | R < Y |
| C2-C3 | ST |
| C3-C4 | R < Y |
| C4-C5 | ST |
| C5-C6 | R < Y |
| C6-S | R ≥ Y |

Here, as shown. in FIG. 5, symbols C1, C2, and so on represent curve change points on the route, respectively. A symbol R represents a curve radius, a symbol Y represents a value of a curve radius set as a threshold value for a gentle curve and a sharp curve on the route, and a symbol ST represents a straight section. A symbol N represents a normal walking section, and a symbol F represents a fast walking section. In the present embodiment, a curve radius R=Y is regarded as a threshold value, a curved section sharper than the threshold value is regarded as a section unsuitable for fast walking, and thus such curve radius and curved section are excluded from fast walking setting during walking pattern setting. The relation between the horizontal line shape of the route and the walking pattern is not limited to the above example, for example, can be defined in advance such that fast walking is not set for a section that is difficult to walk at a fast pace or that may adversely affect legs, such as a continuous S-shaped curve even when the curve has a curve radius R≥Y. Each of the curve change points is a connection point between a straight line and a curved line on the route, and is set as a point where, for example, a curvature being 0 in a straight section changes to a certain. finite value. As a curve radius R in a certain section on the route, an average value of curve radius in such a section or a minimum curve radius can be adopted.

FIG. 6 is a schematic vertical cross-sectional view showing a relation between a distance and an elevation for the walking route in the example of FIG. 5. Referring to FIG. 6, the exemplary route includes several gradient sections together with the curves shown in FIG. 5. The route exemplified in FIG. 6 has the following vertical line shape starting from a spot (a distance being s=0) as a starting point.

| Section on route | Gradient U, D |
|---|---|
| 0-G1 | L |
| G1-G2 | U > X |
| G2-G3 | L |
| G3-G4 | D > X |
| G4-G5 | L |
| G5-S | U ≤ X |

Here, symbols G1, G2, and so on represent gradient change points on a vertical cross-section of the route, respectively. A symbol X represents a value of an inclination angle set as a threshold value for a gentle gradient and a steep gradient, and a symbol L represents a horizontal (flat) section. Symbols U and D represent an upward gradient and a downward gradient, respectively. In the present embodiment, U and D=X are regarded as threshold values for upward gradient and downward gradient, respectively, upward gradient and downward gradient sections steeper than the threshold value are regarded as sections unsuitable for fast walking, and thus are excluded from fast walking setting during walking pattern setting. The relation between the vertical line shape of the route and the walking pattern is not limited to the above example. For example, the relation can be appropriately set by changing the threshold value of the inclination angle X depending on the upward gradient and the downward gradient. Each of the gradient change points is a connection point between a flat part and the gradient on the route. As a gradient in a certain section on the route, an average value of gradients in such a section or the steepest gradient value can be adopted.

The route line shape information can be acquired from the external device by the route line shape information acquisition unit 118, but the route line shape information can also be acquired as data by the exercise assistance device 1 itself when the exercise assistance device 1 includes the sensor unit 17 and the GNSS unit 18. In the case of the circular route as illustrated in FIGS. 5 and 6, for example, when a user who possesses or wears the exercise assistance device 1 including the sensor unit 17 such as a smart watch and the GNSS unit 18 performs a moving such as walking or running along the route, it is possible to acquire the route line shape information including information on curves and gradients of the route based on position coordinates and altitude information of the user detected by the sensor unit 17. Specifically, for example, since the position coordinates of the user can be detected by the positioning signal of the GNSS unit 18 and an atmospheric pressure can be detected by the pressure sensor of the sensor unit 17, it is possible to obtain a relation between the change in the horizontal position. coordinates of the user and the atmospheric pressure from such detection data. The route line shape information acquisition unit 118 can generate the horizontal line shape and the vertical line shape of the route corresponding to FIGS. 5 and 6, using such a relation. Returning to the starting point of the circular route may be detected by returning to a spot indicating the same position information as the position information of the starting point from the output of the GNSS unit 18. When the route line shape information is acquired by the exercise assistance device 1, the user may be allowed to input POI as a point of interest on the route from the input unit 14.

The moving pattern setting unit 119 as a section setting unit has a function of using the acquired route line shape information and the user information to create a walking pattern suitable for the user to execute interval walking exercise on the walking route. The moving pattern setting unit 119 first reads the speed during fast walking and the speed during normal walking which are stored in advance based on gender and age of the user, for example. The moving pattern setting unit 119 uses the speed during fast walking and the speed during normal walking to set a walking pattern along the route in a case of executing interval walking in which fast walking and normal walking are repeated for a predetermined period of time on the route obtained from the route line shape information. Here, the route line shape information is not reflected on the walking pattern. Next, the moving pattern setting unit 119 refers to the route line shape information illustrated in FIGS. 5 and 6, and executes a process of changing the section excluded from the setting of fast walking to normal walking in the walking pattern set on the route according to a basic pattern of interval walking. Thus, when there is a fast walking section set as a steep gradient section or a sharp curve section in the basic pattern, such a section is changed to a normal walking section. The moving pattern setting unit 119 registers the walking pattern, which is thus modified based on the route line shape information, in the server computer 3. During creation of the walking pattern, a certain distance is used as a threshold value, and a distance longer than the threshold value may be set for the fast walking section to increase the exercise effect.

FIG. 7 shows an output display example of a walking pattern created by the moving pattern setting unit 119. Along a schematic horizontal line shape of the route, fast walking sections are indicated by diagonal line patterns, and normal walking sections are indicated by dot patterns. It can be seen in FIGS. 5 and 6 that sections specified as steep gradients and sharp curves exceeding a certain threshold value are designated as normal walking sections. At the same time, even in sections where there are no steep gradients or sharp curves, it can be seen that fast walking sections and normal walking sections are alternately set according to the basic pattern of interval walking. In the example of FIG. 7, the vicinity of a “large tree” designated as POI is also set as a normal walking section. In this way, the moving pattern setting unit 119 can automatically create an effective walking pattern for interval walking in consideration of the line shape of the route.

### <Data Processing by Exercise Assistance Device>

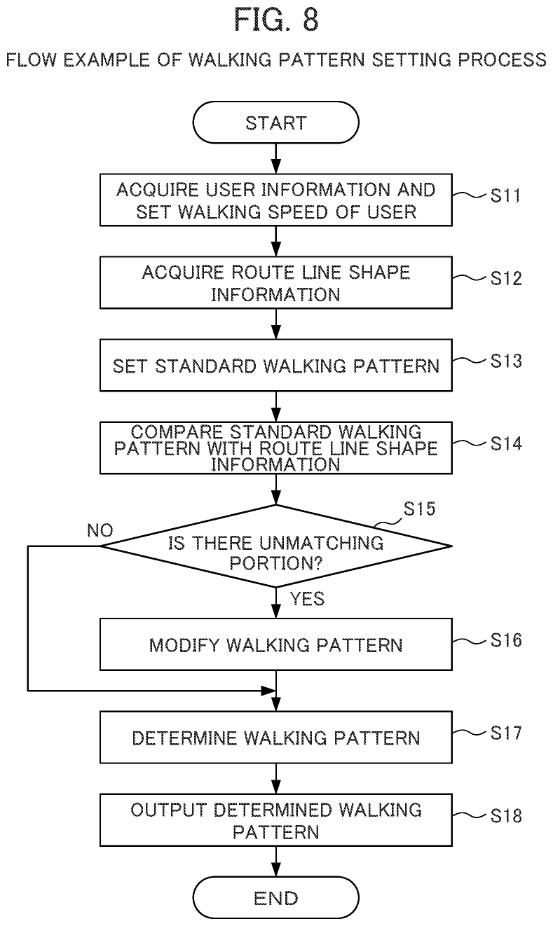

Next, a walking pattern setting process by the exercise assistance device 1 of the present embodiment will be described. FIG. 8 shows a flowchart illustrating a data processing flow of a walking pattern setting process. The walking pattern setting process is data processing for setting a walking pattern suitable for an interval walking, in which an effective exercise effect is obtained by walking as a form of movement of the body, in consideration of a line shape of a walking route. The walking pattern setting process is started by an operation input of a setting start from a user who wants to receive a walking pattern setting service.

The user information acquisition unit 117 acquires user information including ID, age, gender, height, and weight of a user input through the input unit 14, and sets a speed (pace) of fast walking and normal walking suitable for the user, and a repetition time of fast walking and normal walking (Step S11).

The route line shape information acquisition unit 118 acquires, from the external device such as the server computer 3, line shape information of a route along which interval walking exercise is to be performed (Step S12). The route may be configured such that the user can select from existing routes registered in the server computer 3, or may be configured such that a desired route can be set on a map in a map application or the like linked to the exercise assistance device 1 and information on curves, altitudes, POIs, and the like related to the route can be acquired as route line shape information. Alternatively, on a circular route provided in a park or an exercise facility, route line shape information may be generated and used based on altitude information and curve information of the route detected by the sensor unit 17 when a user possessing and wearing the exercise assistance device 1 goes around the circular route.

The moving pattern setting unit 119 sets a standard walking pattern along the target route using interval walking pattern suitable for the user which is set in Step S11 (Step S13). The standard walking pattern is generated based on the speed and repetition time of interval walking of the user set in Step S11, and the general line shape of the route acquired in Step S12, without consideration of the route line shape information.

The moving pattern setting unit 119 compares the standard walking pattern with the route line shape information (Step S14).

The moving pattern setting unit 119 determines whether there is a portion in the standard walking pattern that does not match the route line shape information (Step S15). When it is determined thee there is no unmatching portion (NO in Step S15), the moving pattern setting unit 119 determines the standard walking pattern as the walking pattern for the route to be processed (Step S17). As an example of a route to which the standard walking pattern can be applied as it is, a circular route for logging/walking provided in a flat park with gentle ups and downs and no sharp curves can be considered.

In Step S15, when the moving pattern setting unit 119 determines that there is an unmatching portion between the standard walking pattern and the route line shape information (YES in Step S15), the moving pattern setting unit 119 executes a modification to change the fast walking section set to the section of steep gradients and sharp curves exceeding a predetermined threshold value to the normal walking section (Step S16). In general, such a modification process changes the repetition time between fast walking and normal walking that has been properly secured in the standard walking pattern. The moving pattern setting unit 119 is preferably able to adjust the length of each of the fast walking sections and each of the normal walking sections such that the modified walking pattern is as close to the standard walking pattern as possible. For example, when the normal walking section set to correspond to the sharp curve or the steep gradient is shorter than the standard, it is possible to execute a process of extending the length of the section to approach the length of the standard walking pattern as much as possible.

The walking pattern modified in Step S16 is determined as the walking pattern for the target route in Step S17. The moving pattern setting unit 119 outputs the walking pattern determined in Step S17, and ends the process (Step S18).

### <Modification of Determined Walking Pattern>

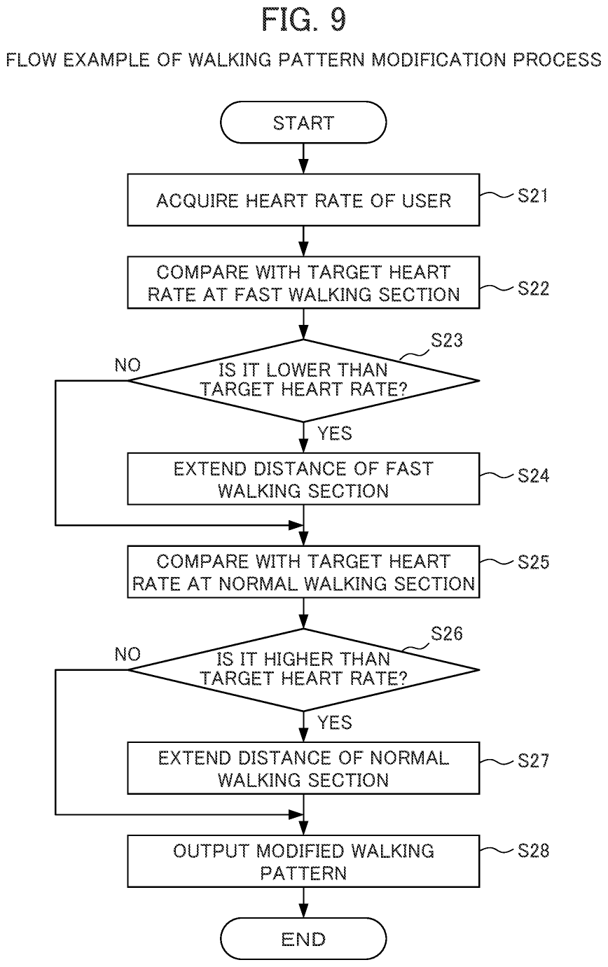

The walking pattern determined by the moving pattern setting unit 119 can be further modified according to results of interval walking actually executed by the user based on the walking pattern. FIG. 9 shows a flowchart showing a process flow example of a walking pattern modification process. The walking pattern modification process is executed by the moving. Pattern setting unit 119, and is started when the user inputs the start of interval walking to the exercise assistance device 1 through the input unit 14.

In the present embodiment, it is possible to add a process of modifying the walking pattern created in consideration of the route line shape information by the exercise assistance device 1, based on heart rate information of the user obtained when the user actually walks along the walking pattern. As described above, it is recommended in interval walking such that using the maximal oxygen uptake (VO2max) of the user (exerciser) as an index, a walking speed is set to exceed 70% of VO2max in the fast walking section and to fall below 40% of VO2max in the normal walking section, for example. The heart rate of the user can similarly be used as a reference of the walking speed. According to the “Karvonen method”, which is widely used to determine the target heart rate during exercise, the target heart rate HR during exercise can be obtained by Formula of HR=(220−age−resting heart rate)×exercise intensity+resting heart rate, and the target heart rate varies depending on the resting heart rate, which differs from person to person, and a desired exercise intensity. In the present embodiment, a model value of the target heart rate during exercise is set in advance according to gender and age of the user, and is used as a reference for modifying the fast walking speed and the normal walking speed.

In the walking pattern modification process illustrated in FIG. 9, the moving pattern setting unit 119 acquires the heart rate of the user from the heartbeat sensor provided in the sensor unit 17 of the exercise assistance device 1 or the terminal device 2 such as a smart watch that cooperates with the exercise assistance device 1 (Step S21). The acquired heart rate data is stored in the main storage unit 12 together with the position information on the route, and the data can be distinguished whether to be data for the fast walking section or to be data for the normal walking section.

First, the moving pattern setting unit 119 compares the acquired heart rate data with the target heart rate in the fast walking section stored in advance in the main storage unit 12, for example (Step S22). When it is determined that the acquired heart rate data is lower than the target heart rate (YES in Step S23), the moving pattern setting unit 119 extends the distance of the corresponding fast walking section such that the heart rate in the same fast walking section reaches the target heart rate (Step S24). The degree of extension of the distance in this case can be determined in advance by a test.

Next, the moving pattern setting unit 119 compares the acquired heart rate data with the target heart rate in the normal walking section stored in advance in the main storage unit 12, for example (Step S25). When it is determined that the acquired heart rate data is higher than the target heart rate (YES in Step S26), the moving pattern setting unit 119 extends the distance of the corresponding normal walking section such that the heart rate in the same normal walking section falls below the target heart rate (Step S27). The degree of extension of the distance in this case can be determined in advance by a test.

The moving pattern setting unit 119 stores the modified walking pattern in the main storage unit 12 or the auxiliary storage unit 13 and outputs it through the output unit 15, thereby ending the walking pattern modification process (Step S28).

As described above, the walking pattern is modified according to the heart rate of the user who performs the interval walking, and thus an effective walking pattern can be set in which the heart rate is raised to obtain the necessary exercise intensity during the fast walking and the heart rate is sufficiently lowered not to overload the body during the normal walking.

Further, during the interval walking, the set walking pattern can be presented to the user visually or acoustically via the output unit 15 or the exercise assistance device 1. The presentation may be performed in combination with a tactile manner such as vibration, and the timing of output may be at the same time as the pattern switching, or may be announced in advance such as “fast walking section coming up”. In addition, the tactile manner can also be used alone as a notification manner. Thus, an effective exercise pattern can be performed. more reliably, and thus it can be expected to bring about an appropriate exercise effect for the user.

### Second Embodiment

A second embodiment of the present disclosure will be described. The exercise assistance device 1 described in the above embodiment has the function of creating the walking pattern when the user performs interval walking in which the normal walking and the fast walking are repeated at predetermined time intervals. An exercise assistance device 1 according to the second embodiment is intended to apply such a function not only to walking but also to general moving of the body including running and jogging. Here, the exercise assistance device 1 implements a function of adjusting and setting a pattern of moving on a route along which the user, that is, the exerciser performs moving, that is, a section for running, jogging, and walking, based on route line shape information.

### <Configuration and Function of Exercise Assistance Device 1 According to Second Embodiment>

### <<Configuration of Exercise Assistance Device 1>>

The exercise assistance device 1 of the present embodiment basically includes a configuration similar to that of the exercise assistance device 1 of the first embodiment. Based on the functional difference from the first embodiment, the moving pattern setting unit provided in the processing unit 11 of the first embodiment sets patterns including not only walking but also running and jogging.

### <<Function of Exercise Assistance Device 1>>

Here, a function of the moving pattern setting unit 119 will be described that implements a moving pattern setting function in the second embodiment. For ease of understanding, it is assumed that the route line shape information for moving such as running is the same as in FIGS. 5 and. 6.

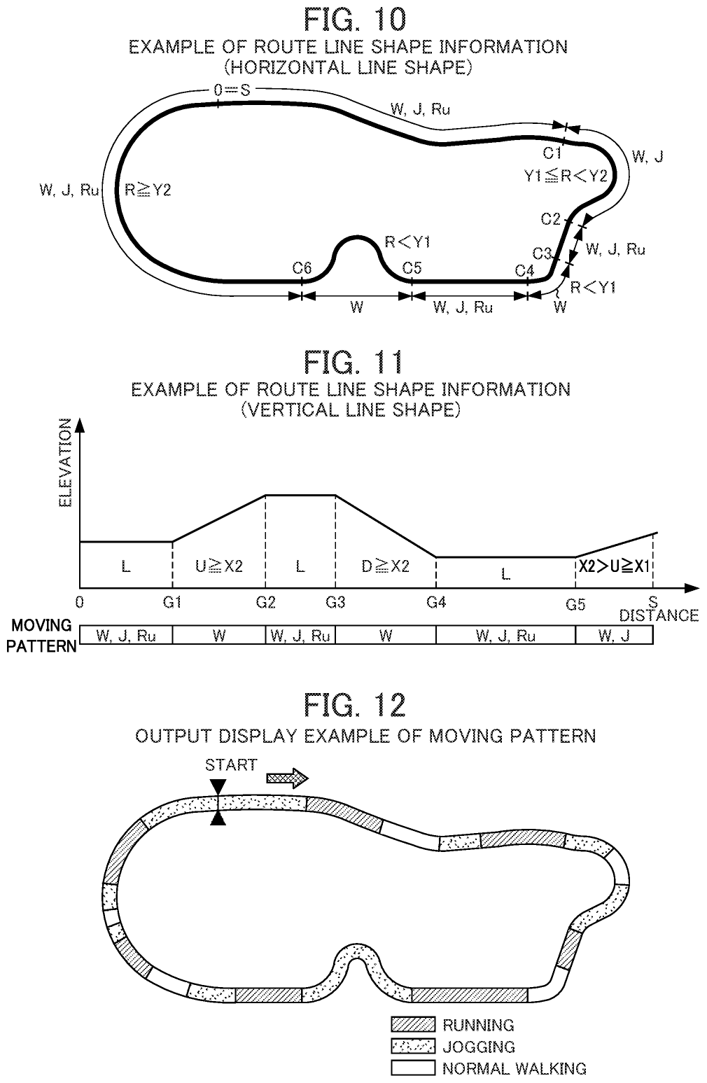

FIG. 10 illustrates a horizontal line shape of a route corresponding to FIG. 5. In the present embodiment, three modes of walking, jogging, and running are assumed as modes of moving, and are denoted by symbols of W, C, and Ru, respectively. Symbols Y1 and Y2 respectively represent threshold values used in a case of classifying curves on the route according to a curve radius. Here, it is assumed that Y2>Y1, that is, the curve radius increases from Y1 toward Y2, that is, becomes gentle. In the second embodiment, using R=Y1, Y2 (Y2>Y1) as a threshold value with respect to a curve in a certain section on the route, it is set such that only walking can be assigned when R<Y1, walking or jogging can be assigned when Y2≥Y1, and walking, jogging, or running can be assigned when R≥Y2. With such setting, walking can be assigned at sharp curves or corners that are difficult to pass at high speed and jogging or running can be additionally assigned at gentler curves, allowing the user to exercise safely on the route.

FIG. 11 illustrates a vertical line shape of a route corresponding to FIG. 6. Symbols X1 and X2 respectively represent threshold values used in a case of classifying gradients on the route according to inclination angles of the gradients. Here, it is assumed that X2>X1, that is, the gradient increases from X1 toward X2, that is, becomes steeper. In the second embodiment, using X1 and X2 (X2>X1) as a threshold value with respect to a relation between the upward gradient U and the downward gradient D on the route and the exercise pattern, it is set such that only walking can be assigned when U, D>X2, walking or jogging can be assigned when X2≥U, D>X1, and walking, jogging, or running can be assigned when X1≥U, D. This prevents jogging or running from being assigned according to the gradient because the burden (impact force) on the legs when running or walking increases as the gradient becomes steeper. Thus, an excessive physical load is prevented when the user is exercising. Regarding the upward gradient U, when the gradient is not too steep, running or logging may rather increase the exercise effect, so it is possible to assign jogging or running by setting an appropriate threshold value.

FIG. 12 shows an output example of exercise patterns created for the routes exemplified in FIGS. 10 and 11 by the exercise assistance device 1 according to the second embodiment. In the example of FIG. 12, along a schematic horizontal line shape of the route, running sections are indicated by diagonal line patterns, logging sections are indicated by dot patterns, and walking sections are indicated by white blanks. It can be seen in FIGS. 10 and 11 that sections specified as steep gradients and sharp curves exceeding a certain threshold value are designated as walking sections. At the same time, even in sections where there are no steep gradients or sharp curves, running, jogging, and walking are arranged according to a predetermined regularity. The distance setting and arrangement of each of the sections of running, logging, and walking can be determined based on training plan formulated to improve exercise effects. Alternatively, the exercise pattern set by the moving pattern setting unit 119 based on the route line shape information may be adopted as it is. In this way, the moving pattern setting unit 119 can automatically create an exercise pattern in consideration of the line shape of the route along which the moving including running and jogging is performed, as in the first embodiment.

### <Data Processing by Exercise Assistance Device According to Second Embodiment>

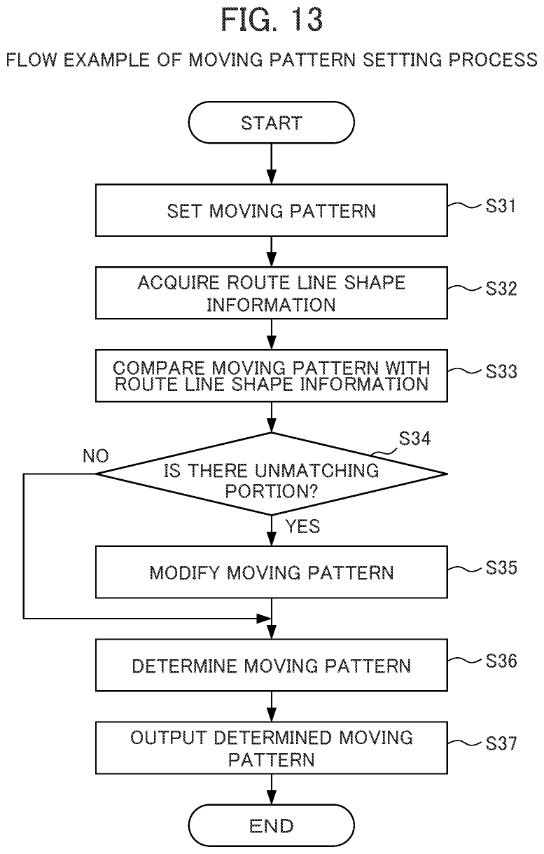

Next, a moving pattern setting process by the exercise assistance device 1 of the second embodiment will be described. FIG. 13 shows a flowchart illustrating a data processing flow of a moving pattern setting process. The moving pattern setting process is data processing for setting a moving pattern suitable for obtaining an effective exercise effect by mainly using running as a form of moving in combination with jogging and walking, in consideration of the line shape of a route. The moving pattern setting process is started by an operation input of a setting start from a user who wants to receive a moving pattern setting service.

The user inputs a moving pattern, which is intended to be performed by the user, through the input unit 14 (Step S31). In the present embodiment, since a moving pattern in so-called interval training is considered to vary widely depending on the purpose, preference, and the like of the user, it is assumed that an moving pattern is given by the user. In this regard, for example, model patterns for interval training may be prepared in the server computer 3 such that the user can select one. The moving pattern set by the user can be set in a form conforming to the standard walking pattern illustrated with reference to FIG. 1 of the first embodiment.

The route line shape information acquisition unit 118 acquires line shape information of a route on which exercise is to be performed from an external device such as a server computer 3 (Step S32). An aspect of acquisition of the route line shape information is the same as in the case of the first embodiment.

The moving pattern setting unit 119 compares the moving pattern set in Step S31 with the route line shape information (Step S33).

The moving pattern setting unit 119 determines whether there is a portion in the set exercise pattern that does not match the route line shape information (Step S34). When it is determined that there is no unmatching portion (NO in Step S34), the moving pattern setting unit 119 determines the moving pattern set in Step S31 as a moving pattern for the route to be processed (Step S36).

In Step 534, when moving pattern setting unit 119 determines that there is an unmatching portion between the initially set exercise pattern and the route line shape information (YES in Step S34), the moving pattern setting unit 119 executes a modification to change running or logging set in the section of gradients and curves exceeding a predetermined threshold value to walking (Step S35). At this time, as in the first embodiment, the moving pattern setting unit 119 is preferably able to adjust the length of each section such that the modified moving pattern is as close to the initially set moving pattern as possible.

The exercise pattern modified in Step S35 is determined as an exercise pattern for the target route in Step S36. The moving pattern setting unit 119 outputs the exercise pattern determined in Step S36, and ends the process (Step S37). In the present embodiment, as in the first embodiment, a process may be executed of modifying the set moving pattern based on biometric information such as a heart rate of the user.

As described above, according to the embodiments of the present disclosure, the exercise assistance device 1 includes: a route line shape information acquisition unit 118 as a route information acquisition unit configured to acquire route information that is information on a route along which a user performs moving; and a section setting unit 119 configured to set, based on the acquired route information, the route to a first section as a section in which the user walks or runs at a first speed and a second section as a section in which the user walks or runs at a second speed lower than the first speed.

Thus, it is possible to create a walking or running pattern that allows the user to enjoy an appropriate exercise effect according to conditions of the route.

The route line shape information acquisition unit 118 may acquire, as the route information, information on a line shape of the route along which the user performs the moving, and the section setting unit 119 may set the first section and the second section based on the information on the line shape of the route along which the user performs the moving.

Thus, it is possible to create a suitable walking or running pattern based on the line shape of the route along which the user walks or runs.

The route line shape information may include gradient information indicating a correspondence relation between a position on the route and a gradient on the route and curve information indicating a correspondence relation between the position on the route and a curve on the route, and the moving pattern setting unit 119 may set the route as a section suitable for the walking or running at the first speed so as to satisfy a condition that the gradient in the section is smaller than a predetermined threshold value and a radius of the curve is larger than a predetermined threshold value.

Thus, it is possible to avoid steep gradients and sharp curves on the route, to set the section in which the moving should be performed faster, and to further enhance the exercise effect of the user.

The moving pattern setting unit 119 may set the first section and the second section on the route such that at least first distance moving at the first speed and at least second distance moving at the second speed are repeated on the route.

Thus, it is possible to create a moving pattern suitable for interval training such as interval walking that enhances the exercise effect of the user.

The moving pattern setting unit 119 may set the first section such that a length of the first section is equal to or greater than a predetermined threshold value.

Thus, it is possible to secure a predetermined distance that gives an appropriate load to the user, as a section for walking or running faster.

The moving pattern setting unit 119 may output the first section and the second section set on the route in a manner that is identifiable through at least one selected from visual, auditory, and tactile senses.

Thus, the user can easily grasp a walking or running pattern created for the route of a certain moving through at least one of visual, auditory, and tactile senses.

The moving may include at least one of walking of the user and running of the user.

Thus, it is possible to provide a pattern of moving, such as walking or running, according to the needs of the user.

The route line shape information acquisition unit 118 may acquire the route line shape information on the route, based on time-series position information of the user detected along the route while the user performs the moving along the route.

Thus, when the user walks or runs on the route for moving such as a circular route, it is possible to acquire the route line shape information.

A place of interest on the route may be recorded in the route line shape information, and the section setting unit may set a vicinity of the place of interest on the route to the second section.

Thus, the user can be encouraged to walk or run more slowly near a place of interest on the route.

The moving pattern setting unit 119 may adjust the set distance of the first section and/or the second section, based on biometric -information acquired about the user.

Thus, based on the change in biometric information. such as a heart rate of the user, it is possible to set the distance of the faster walking or running section and the distance of the slower walking or running section so as to enhance the exercise effect for the user.

The processing sequence described above can be executed by hardware, and can also be executed by software. In other words, the functional configuration of FIG. 3 is merely an illustrative example, and the present invention is not particularly limited thereto. More specifically, the types of functional blocks employed to realize the above-described functions are not particularly limited to the examples shown in FIG. 3, so long as the exercise assistance device 1 can be provided with the functions enabling the aforementioned processing sequence to be executed in its entirety. In addition, a single functional block may be configured by a single piece of hardware, a single installation of software, or a combination thereof. The functional configurations of the present embodiment are realized by a processor executing arithmetic processing, and processors that can be used for the present embodiment include a unit configured by a single unit of a variety of single processing devices such as a single processor, multi-processor, multi-core processor, etc., and a unit in which the variety of processing devices are combined with a processing circuit such as ASIC (Application Specific Integrated Circuit) or FPGA (Field-Programmable Gate Array).

In the case of having the series of processing executed by software, the program constituting this software is installed from a network or recording medium to a computer or the like. The computer may be a computer equipped with dedicated hardware. In addition, the computer may be a computer capable of executing various functions, e.g., a general-purpose personal computer, by installing various programs.

The storage medium containing such a program can not only be constituted by the removable medium such as USB memory distributed separately from the device main body for supplying the program to a user, but also can be constituted by a storage medium or the like supplied to the user in a state incorporated in the device main body in advance. The removable medium is composed of, for example, a magnetic disk (including a floppy disk), an optical disk, a magnetic optical disk, or the like. The optical disk is composed of, for example, a CD-ROM (Compact Disk-Read Only Memory), a DVD (Digital Versatile Disk), Blu-ray (Registered Trademark) or the like. The magnetic optical disk is composed of an MD (Mini-Disk) or the like. The storage medium supplied to the user in a state incorporated in the device main body in advance is constituted by, for example, the ROM in which the program is recorded or a hard disk included in the auxiliary storage unit 13, etc.

It should he noted that, in the present specification, the steps defining the program recorded in the storage medium include not only the processing executed in a time series following this order, but also processing executed in parallel or individually, which is not necessarily executed in a time series.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An information processing device comprising:
a route information acquisition unit configured to acquire route information that is information on a route along which a user performs at least two types of moving with different moving speeds; and
a section setting unit configured to set, on the route based on the acquired route information, a first section as a section in which the user performs first moving at a first speed and a second section as a section in which the user performs second moving at a second speed lower than the first speed.

2. The information processing device according to claim 1, wherein the route information acquisition unit acquires, as the route information, information on a line shape of the route along which the user performs the moving, and the section setting unit sets the first section and the second section based on the information on the line shape of the route along which the user performs the moving.

3. The information processing device according to claim 1, wherein the route information includes gradient information indicating a correspondence relation between a position on the route and information on a gradient on the route and curve information indicating a correspondence relation between the position on the route and information on a curve on the route, and the section setting unit sets on the route a section suitable for the first moving so as to satisfy a condition that the gradient in the section is smaller than a predetermined threshold value and a radius of the curve is larger than a predetermined threshold value.

4. The information processing device according to claim 1, wherein the section setting unit sets the first section and the second section on the route such that the first moving and the second moving are repeated on the route.

5. The information processing device according to claim 1, wherein the section setting unit gets the first section such that a length of the first section is equal to or greater than a predetermined threshold value.

6. The information processing device according to claim 1, wherein the section setting unit outputs information indicating the first section and the second section set on the route in a manner that is identifiable through at least one selected from visual, auditory, and tactile senses.

7. The information processing device according to claim 1, wherein the at least two types of moving include walking and fast walking, or walking, jogging, and running.

8. The information processing device according to claim 1, wherein the route information acquisition unit acquires route information. on the route, based on time-series position information of the user detected along the route while the user performs the moving along the route.

9. The information processing device according to claim 1, wherein a place of interest on the route is recorded in the route information, and the section setting unit sets a vicinity of the place of interest on the route to the second section.

10. The information processing device according to claim 1, wherein the section setting unit adjusts a distance of the first section or the second section set on the route, based on biometric information acquired while the user performs the moving.

11. An information processing method executed by an information processing device, the method comprising:
acquiring route information that is information on a route along which a user performs at least two types of moving with different moving speeds; and
setting, on the route based on the acquired route information, a first section as a section in which the user performs first moving at a first speed and a second section as a section in which the user performs second moving at a second speed lower than the first speed.

12. The information processing method according to claim 11, wherein the route information includes gradient information indicating a correspondence relation between a position on the route and information on a gradient on the route and curve information. indicating a correspondence relation between the position on the route and information on a curve on the route, and the setting the route includes setting on the route a section suitable for the first moving so as to satisfy a condition that the gradient in the section is smaller than a predetermined threshold value and a radius of the curve is larger than a predetermined threshold value.

13. The information processing method according to clam 11, wherein the setting the route includes setting the first section and the second section on the route such that the first moving and the second moving are repeated on the route.

14. The information processing method according to claim 11, wherein the at least two types of moving include walking and fast walking, or walking, jogging, and running.

15. The information processing method according to claim 11, wherein the setting the route includes adjusting a distance of the first section or the second section set on the route, based on biometric information acquired while the user performs the moving.

16. A non-transitory recording medium storing a computer-readable program of an information processing device,
the information processing device including:
a storage unit configured to store the program; and a processing unit configured to, based on the program, execute:
acquiring route information that is information on a route along which a user performs at least two types of moving with different moving speeds; and
setting, on the route based on the acquired route information, a first section as a section in which the user performs first moving at a first speed and a second section as a section in which the user performs second moving at a second speed lower than the first speed.

17. The recording medium according to claim 16, wherein the route information includes gradient information indicating a correspondence relation between a position on the route and information on a gradient on the route and curve information indicating a correspondence relation between the position on the route and information on a curve on the route, and the setting the route includes setting on the route a section suitable for the first moving so as to satisfy a condition that the gradient in the section is smaller than a predetermined threshold value and a radius of the curve is larger than a predetermined threshold value.

18. The recording medium according to claim 16, wherein the setting the route includes setting the first section and the second section on the route such that the first moving and the second moving are repeated on the route.

19. The recording medium according to claim 16, wherein the at least two types of moving include walking and fast walking, or walking, jogging, and running.

20. The recording medium according to claim 16, wherein the setting the route includes adjusting a distance of the first section or the second section set in the route, based on biometric information acquired while the user performs the moving.
